(19) United States
(12) Patent Application Publication
(10) Pub. No.: US 2011/0033886 A1
(43) Pub. Date: Feb. 10, 2011

(54) GDF-15 AS BIOMARKER IN TYPE 1 DIABETES

(76) Inventors: Georg Hess, Mainz (DE); Andrea Horsch, Mannheim (DE); Dietmar Zdunek, Tutzing (DE)

Correspondence Address: ROCHE DIAGNOSTICS OPERATIONS INC., 9115 Hague Road, Indianapolis, IN, 46250-0457, US

(21) Appl. No.: 12/908,987

(22) Filed: Oct. 21, 2010

Related U.S. Application Data
(63) Continuation of application No. PCT/EP2009/056090, filed on May 19, 2009.

(30) Foreign Application Priority Data
May 20, 2008 (EP) 08156537.6

(51) Int. Cl. C12Q 1/02; C12M 1/34

(52) U.S. Cl. 435/29; 435/287.1

(57) ABSTRACT

The present invention relates to a method of predicting if a diabetes type 1 patient will suffer from one or more complications selected from cardiovascular complications, terminal renal failure, and death, the method including (a) determining the amount of GDF-15 in a sample of a diabetes type 1 patient; and (b) comparing the amount of GDF-15 determined in step (a) to a reference amount and establishing a prediction. Also encompassed by the present invention are devices and kits for carrying out the aforementioned methods.

### RELATED APPLICATIONS

This application is a continuation of PCT/EP2009/056090 filed May 19, 2009 and claims priority to EP 08156537.6 filed May 20, 2008.

### FIELD OF THE INVENTION

The present invention relates to a method for predicting or assessing the risk of a type 1 diabetes patient to suffer from a cardiovascular event and/or terminal renal failure and/or death. The method is based on the determination of growth-differentiation factor-15 (GDF-15) in a sample of a subject suffering from type 1 diabetes. Moreover, the present invention, pertains to a method for predicting the risk of a cardiovascular event, mortality or terminal renal failure for a subject suffering from type 1 diabetes based on the determination of GDF-15 in a sample of the subject. Also encompassed by the present invention are devices and kits for carrying out the aforementioned methods.

### BACKGROUND OF THE INVENTION

An aim of modern medicine is to provide personalized or individualized treatment regimens. Those are treatment regimens which take into account a patient's individual needs or risks. Personalized or individual treatment regimens shall be also taken into account for emergency measures. Specifically, in the case of acute cardiovascular events, a decision for a certain treatment regimen must be made, usually, within a short period of time. Cardiovascular complications, particularly heart diseases, are the leading cause of morbidity and mortality in the Western hemisphere. Cardiovascular complications can remain asymptomatic for long periods of time. However, they may have severe consequences once an acute cardiovascular event, such as myocardial infarction, as a cause of the cardiovascular complication occurs.

There are two main categories of diabetes mellitus type 1 and type 2, which can be distinguished by a combination of features known to the person skilled in the art.

In type 1 diabetes (previously called juvenile-onset or insulin-dependent), insulin production is absent because of autoimmune pancreatic beta-cell destruction possibly triggered by environmental exposure in genetically susceptible people. Destruction progresses subclinically over months or years until beta-cell mass decreases to the point that insulin concentrations are no longer adequate to control plasma glucose levels. The type 1 diabetes generally develops in childhood or adolescence and until recently was the most common form diagnosed before age 30; however, it can also develop in adults.

In type 2 diabetes (previously called adult-onset or non-insulin-dependent), insulin secretion is inadequate. Often insulin levels are very high, especially early in the disease, but peripheral insulin resistance and increased hepatic production of glucose makes insulin levels inadequate to normalized plasma glucose levels. Insulin production then falls, further exacerbating hyperglycemia. The disease generally develops in adults and becomes more common with age. Plasma glucose levels reach higher levels after eating in older than in younger adults, especially after high carbohydrate loads, and take longer to return to normal, in part because of increased accumulation of visceral and abdominal fat and decreased muscle mass.

Chronic kidney disease may result from any cause of renal dysfunction of sufficient magnitude. The most common call in the US is diabetic nephropathy, followed by hypertensive nephroangiosclerosis and various primary and secondary glomerulopathies. A chronic kidney disease (chronic renal failure) is long-standing, progressive deterioration of a renal function. Symptoms develop slowly and include anorexia, nausea, vomiting, stomatitis, dysgeusia, nocturia, lassitude, fatigue, proritus, decreased mental accuity, muscle twitches and cramps, water retention, undernutrition, ulceration and bleeding, peripheral neuropathies, and seizures. Diagnosis is based on laboratory testing of renal function, sometimes followed by renal biopsy.

The conventional diagnostic techniques for cardiovascular complications and their prediction include electrocardiographic and echocardiographic measurements, analysis of symptoms and previous medical history of the patient, such as chest pain, and analysis of some clinical parameters. Recently, these conventional techniques have been further strengthened by the analysis of biomarkers and, in particular, by the analysis of the levels for cardiac Troponins in blood samples of emergency patients. Moreover, natriuretic peptides are also described as suitable biomarkers for diagnosing cardiovascular complications. Even more recently, GDF-15 has been suggested to be an indicator for cardiovascular complications, too (US2003/0232385; Kempf 2006, Circ Res 98: 351-360). Growth-differentiation factor-15 (GDF-15) is a member of the transforming growth factor-βcytokine superfamily. GDF-15 was first identified as macrophage-inhibitory cytokine-1 (MIC-1), and later also named placental transforming growth factor-β(Bootcov 1997, Proc Natl Acad Sci 94:11514-11519; Tan 2000, Proc Natl Acad Sci 97:109-114). It has recently been shown that cultured cardiomyocytes express and secrete GDF-15 via nitric oxide and nitrosative stress-dependent signaling pathways when subjected to simulated ischemia and reperfusion. Moreover, it has been observed in a mouse model of myocardial ischemia and reperfusion injury that GDF-15 expression levels rapidly increase in the ischemic area following coronary artery ligation, and remain elevated in the reperfused myocardium for several days (Kempf loc. cit.).

The conventional diagnostic techniques, specifically for emergency situations, usually do not allow for a reliable diagnosis and/or risk assessment. Thus, based on said diagnostic techniques, a personalized risk prediction can not be determined with sufficient accuracy. As a consequence thereof, for many patients a prediction will be established which is insufficient or which may have adverse side effects.

Therefore, there is a need for diagnostic or prognostic measures which allow an individual risk prediction for a type 1 diabetes patient who is suspicious to suffer from a cardiovascular complication, terminal renal failure, or death, and who may be in need for a certain treatment regimen. Furthermore, there is a need for a reliable general risk prediction or assessment including the risk for mortality in type 1 diabetes patients. In this type of patients, death may result from cardiovascular complications and/or renal failure, or from another reason.

The technical problem underlying the present invention can be seen as the provision of means and methods for complying with the aforementioned needs.

The technical problem is solved by the embodiments characterized in the claims and herein below.

### SUMMARY OF THE INVENTION

Accordingly, the present invention relates to a method of predicting if a diabetes type 1 patient will suffer from one or more complications selected from cardiovascular complications, terminal renal failure, and death, the method comprising
a) determining the amount of GDF-15 in a sample of a diabetes type 1 patient; and
b) comparing the amount of GDF-15 determined in step a) to a reference amount and establishing a prediction.

The method of the present invention, preferably, is an in vitro method. Moreover, it may comprise steps in addition to those explicitly mentioned above. For example, further steps may relate to sample pre-treatments or evaluation of the results obtained by the method. The method of the present invention may be also used for monitoring, confirmation, and subclassification of a type 1 diabetes patient in respect to the complications subject in need of a cardiac intervention. The method may be carried out manually or assisted by automation. Preferably, step (a) and/or (b) may in total or in part be assisted by automation, e.g., by a suitable robotic and sensory equipment for the determination in step (a) or a computer-implemented comparison in step (b).

### DETAILED DESCRIPTION OF THE INVENTION

The term “predicting” as used herein refers to assessing the probability according to which a type 1 diabetes patient will suffer from one or more of a cardiovascular complication, terminal renal failure and death (i.e. mortality) within a defined time window (predictive window) in the future. The mortality may be caused by the cardiovascular complication. The predictive window is an interval in which the subject will develop one or more of the complications according to the predicted probability. The predictive window may be the entire remaining lifespan of the subject upon analysis by the method of the present invention. Preferably, however, the predictive window is an interval of one month, six months or one, two, three, four, five oaten years after appearance of the cardiovascular complication (more preferably and precisely, after the sample to be analyzed by the method of the present invention has been obtained).

As will be understood by those skilled in the art, such an assessment is usually not intended to be correct for 100% of the subjects to be analyzed. The term, however, requires that the assessment will be valid for a statistically significant portion of the subjects to be analyzed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the prediction will be correct for at least 60%, at least 70%, at least 80%, or at least 90% of the subjects of a given cohort.

The term “patient” or “subject” as used herein relates to animals, preferably mammals, and, more preferably, humans.

It is envisaged in accordance with the aforementioned method of the present invention that the subject shall suffer from type 1 diabetes. The subject thus exhibits the signs of diabetes which are known to the person skilled in the art and which have, partly, been laid out beforehand, see introductory part.

Years of poorly controlled diabetes lead to multiple, primarily vascular complications that may affect both small (microvascular) and large (macrovascular) vessels. Microvascular disease underlies the three most common and devastating manifestations of diabetes mellitus: retinopathy, nephropathy, and neuropathy.

Diabetic nephropathy is a leading cause of chronic renal failure. It is characterized by thickening of the glomerula basement membrane, mesangial expansion, and glomerula sclerosis. These changes cause glomerula hypertension and progressive decline. Systemic hypertension may accelerate progression. The disease is usually asymptomatic until a nephrotic syndrome or renal failure develops.

Macrovascular disease (large-vessel atherosclerosis) is a result of the hyperinsulinemia, dyslipidemia, and hyperglycemia characteristic of diabetes. Manifestations are angina pectoris and myocardial infarction, transient ischemic attacks and strokes, and peripheral arterial disease. Diabetic cardiomyopathy is thought to result from many factors, including epicardial atherosclerosis, hypertension and left ventricular hypertrophy, microvascular disease, endothelial and autonomic dysfunction, obesity, and metabolic disturbances. Patients develop heart failure due to impairment in left ventricular systolic and diastolic function and are more likely to develop heart failure after myocardial infarction.

Chronic renal failure can be roughly categorized as diminished renal reserve, renal insufficiency, or renal failure (end-stage renal disease). Initially, as renal tissue loses function, there are few abnormabilities because the remaining tissue increases its performance. Decrease renal function interferes with the kidneys' abilities to maintain fluid and electrolyc homeostasis.

The diagnosis of renal failure includes the determination of serum creatinin levels. When creatinin levels rise, chronic renal failure is usually first suspected. The initial step is to determine whether the renal failure is acute, chronic, or acute superimposed on chronic (i.e. an acute disease that further compromises renal function in a patient with chronic renal failure). The cause of renal failure is also determined. Sometimes determining a duration of renal failure helps determine the cause. Testing includes urine analysis with examination of the urinary sediment, electrolytes, urea nitrogen, and creatinin, phosphate, calcium. Sometimes specific serologic tests inhibit to determine the cause. Urine analysis findings depend on the nature of the underlying disorder, but broad or especially waxy casts often are prominent in advanced renal failure of any cause. An ultrasound examination of the kidneys is usually helpful in evaluating for obstructive uropathy and in distinguishing acute from chronic renal failure based on kidney size. Except in'certain conditions, patients with chronic renal failure have small shrunken kidneys with thinned, hyperechoic cortex. Obtaining a precise diagnosis becomes increasingly difficult as renal function reaches values close to those of end-stage renal disease. The definite diagnostic tool is renal biopsy, but it is not recommended when ultrasonography indicates small, or fibrotic kidneys.

Progression of chronic renal failure is predicted in most cases by the degree of proteinuria. Patients with nephrotic-range proteinuria usually have a poorer prognosis and progress to renal failure more rapidly. Progression may occur even if the underlying disorder is not active. Hypertension is associated with more rapid progression as well.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

The term “Growth-Differentiation Factor-15” or “GDF-15” relates to a polypeptide being a member of the transforming growth factor (TGF)-β cytokine superfamily. The terms polypeptide, peptide and protein are used interchangeable throughout this specification. GDF-15 was originally cloned as macrophage-inhibitory cytokine-1 and later also identified as placental transforming growth factor-β, placental bone morphogenetic protein, non-steroidal anti-inflammatory drug-activated gene-1, and prostate-derived factor (Bootcov loc cit; Hromas, 1997 Biochim Biophys Acta 1354:40-44; Lawton 1997, Gene 203:17-26; Yokoyama-Kobayashi 1997, J Biochem (Tokyo), 122:622-626; Paralkar 1998, J Biol Chem 273:13760-13767). Similar to other TGF-β-related cytokines, GDF-15 is synthesized as an inactive precursor protein, which undergoes disulfide-linked homodimerization. Upon proteolytic cleavage of the N-terminal pro-peptide, GDF-15 is secreted as a ˜28 kDa dimeric protein (Baùskin 2000, Embo J 19:2212-2220). Amino acid sequences for GDF-15 are disclosed in WO99/06445, WO00/70051, WO2005/113585, Bonner 1999, Gene 237: 105-111, Bootcov loc. cit, Tan loc. cit., Baek 2001, Mol Pharmacol 59: 901-908, Hromas loc cit, Paralkar loc cit, Morrish 1996, Placenta 17:431-441 or Yokoyama-Kobayashi loc cit. GDF-15 as used herein encompasses also variants of the aforementioned specific GDF-15 polypeptides. Such variants have at least the same essential biological and immunological properties as the specific GDF-15 polypeptides. In particular, they share the same essential biological and immunological properties if they are detectable by the same specific assays referred to in this specification, e.g., by ELISA assays using polyclonal or monoclonal antibodies specifically recognizing the GDF-15 polypeptides. A preferred assay is described in the accompanying Examples. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific GDF-15 polypeptides. The degree of identity between two amino acid sequences can be determined by algorithms well known in the art. Preferably, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, Wis.), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. Variants referred to above may be allelic variants or any other species specific homologs, paralogs, or orthologs. Moreover, the variants referred to herein include fragments of the specific GDF-15 polypeptides or the aforementioned types of variants as long as these fragments have the essential immunological and biological properties as referred to above. Such fragments may be, e.g., degradation products of the GDF-15 polypeptides. Further included are variants which differ due to posttranslational modifications such as phosphorylation or myristylation.

Determining the amount of GDF-15 or any other peptide or polypeptide referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g., reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on ELECSYS analyzers, Roche Diagnostics, GMBH), CBA (an enzymatic cobalt binding assay, available for example on Roche-Hitachi analyzers), and latex agglutination assays (available for example on Roche-Hitachi analyzers).

Preferably, determining the amount of a peptide or polypeptide comprises the steps of (a) contacting a cell capable of eliciting a cellular response the intensity of which is indicative of the amount of the peptide or polypeptide with the peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g., a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

Determining the amount of a peptide or polypeptide may, preferably, comprises the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g., nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g., phage display. Antibodies as referred to herein include both polyclonal and monoclonal antibodies, as well as fragments thereof, such as Fv, Fab and F(ab)₂ fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g., according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g., by NMR or surface plasmon resonance.

Second, if the ligand also serves as a substrate of an enzymatic activity of the peptide or polypeptide of interest, an enzymatic reaction product may be measured (e.g., the amount of a protease can be measured by measuring the amount of cleaved substrate, e.g., on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled, with a detectable label prior to the reaction. Preferably, the sample is contacted with the substrate for an adequate period of time. An adequate period of time refers to the time necessary for an detectable, preferably measurable, amount of product to be produced. Instead of measuring the amount of product, the time necessary for appearance of a given (e.g., detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (“e.g., magnetic beads”, including paramagnetic and superparamagnetic labels), and fluorescent labels. Enzymatically active labels include e.g., horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star (Amersham Biosciences), ECF (Amersham Biosciences). A suitable enzyme-substrate combination may result in a colored reaction product, fluorescence or chemiluminescence, which can be measured according to methods known in the art (e.g., using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g., Alexa 568). Further fluorescent labels are available e.g., from Molecular Probes (Oregon). Also the use of quantum dots as fluorescent labels is contemplated. Typical radioactive labels include ³⁵S, ¹²⁵I, ³²P, ³³P and the like. A radioactive label can be detected by any method known and appropriate, e.g., a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamid gel electrophoresis (SDS-PAGE), Western Blotting, and mass spectrometry), can be used alone or in combination with labeling or other detection methods as described above.

The amount of a peptide or polypeptide may be, also preferably, determined as follows: (a) contacting a solid support comprising a ligand for the peptide or polypeptide as specified above with a sample comprising the peptide or polypeptide and (b) measuring the amount peptide or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20(1):9-12). In such suspension arrays, the carrier, e.g., a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide or peptide, the relative amount or concentration of the polypeptide or peptide as well as any value or parameter which correlates thereto or can be derived therefrom. Such values or parameters comprise intensity signal values from all specific physical or chemical properties obtained from the peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison referred to in step (b) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e. automatically provide the desired assessment in a suitable output format. Based on the comparison of the amount determined in step a) and the reference amount, it is possible to predict the risk of the subject of suffering of one or more of the complications referred to herein. Therefore, the reference amount is to be chosen so that either a difference or a similarity in the compared amounts allows identifying those diabetes type I patients which are at risk of suffering of one or more of the complications referred to herein, and which are not.

Accordingly, the term “reference amount” as used herein refers to an amount which allows predicting whether a diabetes type 1 patients is at risk of suffering from one or more of a cardiovascular complication, terminal renal failure, and death. Accordingly, the reference may either be derived from (i) a type 1 diabetes patient known to have suffered from one or more of the complications, or (ii) a type 1 diabetes patient known to have not suffered from the complications. Moreover, the reference amount may define a threshold amount, whereby an amount larger than the threshold shall be indicative for a subject at risk to develop one or more of the complications while an amount lower than the threshold amount shall be an indicator for a subject not at risk to develop the complications. The reference amount applicable for an individual subject may vary depending on various physiological parameters such as age, gender, or subpopulation, as well as on the means used for the determination of the polypeptide or peptide referred to herein. A suitable reference amount may be determined by the method of the present invention from a reference sample to be analyzed together, i.e. simultaneously or subsequently, with the test sample. A preferred reference amount serving as a threshold may be derived from the upper limit of normal (ULN), i.e. the upper limit of the physiological amount to be found in a population of apparently healthy subjects. The ULN for a given population of subjects can be determined by various well known techniques. A suitable technique may be to determine the median of the population for the peptide or polypeptide amounts to be determined in the method of the present invention.

In a preferred embodiment of the method of the present invention, the reference amount (i.e. the threshold amount) for GDF-15 is 1500 pg/ml, preferably 2000 pg/ml, more preferably 2500 pg/ml, still more preferably 3000 pg/ml, most preferably 3500 pg/ml.

An amount of GDF-15 higher than the reference is indicative for a subject being at risk of developing one or more of the complications.

On the other hand, an amount of GDF-15 of below or equal to 1500 pg/ml, more preferably below or equal to 1000 pg/ml, most preferably below or equal to 500 pg/ml is indicative for a subject being at low risk or not at risk of developing one or more of the complications.

Advantageously, it has been found in the study underlying the present invention that GDF-15 is a reliable prognostic biomarker for predicting the risk of a type 1 diabetes patient to suffer from one or more complications selected from cardiovascular complications, terminal renal failure, and death. Thanks to the present invention, a risk stratification can be easily performed, allowing to initiate medical, physical or dietary treatments of the patient, including adapting the patient's lifestyle. In case the patients' risk turns out to be non existent or low, a time and/or cost intensive or, as the case may be, dangerous therapy can be avoided. Thus, the method of the present invention will be beneficial for the health system in that resources will be saved. It is to be understood that according to the method of the present invention described herein above and below, the amount of GDF-15 or means for the determination thereof can be used for the manufacture of a diagnostic composition for identifying a subject being susceptible for a cardiac intervention.

In the context of the present invention, the term “cardiovascular complication” refers to acute cardiovascular events and to chronic cardiovascular diseases. In the context of the present invention, acute events are more often observed than chronic diseases.

Acute cardiovascular events are, preferably, stroke or acute coronary syndromes (ACS). ACS patients can show unstable angina pectoris (UAP) or myocardial infarction (MI). MI can be an ST-elevation MI (STEMI) or a non-ST-elevated MI (NSTEMI). The occurring of an ACS can be followed by a left ventricular dysfunction (LVD) and symptoms of heart failure.

A chronic disorder of the cardiovascular system as used herein encompasses coronary heart diseases, stable angina pectoris (SAP) or heart failure, preferably chronic heart failure The term “heart failure (HF)” as used herein refers to an impaired systolic and/or diastolic function of the heart. Preferably, the term relates to congestive heart failure which may be caused by various underlying diseases or disorders. Preferably, heart failure referred to herein is also chronic heart failure. Heart failure can be classified into a functional classification system according to the New York Heart Association (NYHA). Patients of NYHA Class I have no obvious symptoms of cardiovascular disease but already have objective evidence of functional impairment. Physical activity is not limited, and ordinary physical activity does not cause undue fatigue, palpitation, or dyspnea (shortness of breath). Patients of NYHA class II have slight limitation of physical activity. They are comfortable at rest, but ordinary physical activity results in fatigue, palpitation, or dyspnea. Patients of NYHA class III show a marked limitation of physical activity. They are comfortable at rest, but less than ordinary activity causes fatigue, palpitation, or dyspnea. Patients of NYHA class IV are unable to carry out any physical activity without discomfort. They show symptoms of cardiac insufficiency at rest.

It is to be understood that the subject to be identified by the aforementioned method, preferably, has objective evidence of impaired systolic and/or diastolic function of the heart as shown, for example, by echocardiography, angiography, scintigraphy, or magnetic resonance imaging. This functional impairment can be accompanied by symptoms of heart failure as outlined above (NYHA class II-IV), although some patients may present without significant symptoms (NYHA I).

Terminal renal failure, in general, can be seen as diabetic nephropathy with a progredient renal function deterioration (raise in creatinine levels and other urinary excreted substances). The end stadium is terminal renal failure, wherein the kidneys excrete only low amounts of urine or no urine at all. Caused by the retention of the urinary excreted substances, the individual needs to be subjected to dialysis, which may be overcome by kidney transplantation. Kidney transplantation, however, suffers from the drawback that the new kidney will also be attacked by nephropathy; furthermore, by the immunosuppressive therapy, the adaptation of the individual to diabetes mellitus is hampered.

The term “mortality” as used herein relates to any kind of mortality, in particular mortality which is caused by the cardiovascular complication, e.g., as a result of myocardial (re-)infarction, heart failure, stroke, or by terminal renal failure.

The present invention, furthermore, relates to a method of assessing the risk of a diabetes type 1 patient to suffer from one or more complications selected from cardiovascular complications, terminal renal failure, and death, the method comprising
a) determining the amount of GDF-15 in a sample of a diabetes type 1 patient; and
b) comparing the amount of GDF-15 determined in step a) to a reference amount, thereby assessing the risk.

The term “assessing the risk” as used herein means estimating the probability whether a subject will in the future suffer from a cardiovascular complication, renal failure, and/or death, or not. As will be understood by those skilled in the art, the assessment underlying the invention is usually not intended to be correct for all (i.e. 100%) of the subjects to be identified. The term, however, requires that a statistically significant portion of subjects can be identified (e.g., a cohort in a cohort study). Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. More preferably, at least 60%, at least 70%, at least 80% or at least 90% of the subjects of a population can be properly identified by the method of the present invention.

In a preferred embodiment of the method of the present invention, the reference amount (i.e. the threshold amount) for GDF-15 is 1500 pg/ml, preferably 2000 pg/ml, more preferably 2500 pg/ml, still more preferably 3000 pg/ml, most preferably 3500 pg/ml.

An amount of GDF-15 larger than the reference is indicative for an elevated risk of mortality or a further acute cardiovascular event.

On the other hand, an amount of GDF-15 of below or equal to 1500 pg/ml, more preferably below or equal to 1000 pg/ml, most preferably below or equal to 500 pg/ml indicates that the risk of mortality or a further acute cardiovascular event is low or can be excluded.

The expression “assessing the risk of suffering from a complication/mortality” as used herein means that the subject (i.e. a type 1 diabetes patient) to be analyzed by the method of the present invention is allocated either into the group of subjects of a population having a normal, i.e. non-elevated, risk for the complications or mortality, or into a group of subjects having a significantly elevated risk. An elevated risk as referred to in accordance with the present invention means that the risk of complication/mortality within a predetermined predictive window is elevated significantly for a subject with respect to the average risk for complication/mortality in a population of subjects.

In principle, it has been found that GDF-15 or means for determining the amount of GDF-15 can be used for the manufacture of a diagnostic composition for predicting whether a type 1 diabetic patient is at risk of a complication/mortality.

The present invention further relates to a method of deciding on the administration of medicaments in a diabetes type 1 patient being susceptible to suffer from a cardiovascular complication, terminal renal failure, and/or death, the method comprising
a) determining the amount of GDF-15 in a sample of a diabetes type 1 patient;
b) comparing the amount of GDF-15 determined in step a) to a reference amount;
c) deciding on the administration.

Preferably; the therapy to be selected for a subject by the method of the present invention said therapy is a drug-based therapy. More preferably, the medicament is an ACE inhibitor, preferably captopril, enalapril, fosinopril, lisinopril, perindopril, quinapril, ramipril, or trandolapril, an AT-1 receptor blocking agent, preferably, candesartan, losartan, or valsartan, a β-receptor blocking agent, preferably, bisoprolol, carvedilol, metoprolol or succinate, or an aldosterone antagonist, preferably, spironolacton or eplerenone.

Another preferred therapy to be selected for a subject in accordance with the present invention is an interventional therapy. An interventional therapy as referred to herein is a therapy which is based on physical interventions with the subject, e.g., by surgery and/or electrophysiological interventions. More preferably, said interventional therapy is cardiac resynchronisation therapy (CRT) or based on implantation of a cardioverter defibrillator (ICD).

Advantageously, by determining the GDF-15 amount in a sample of a subject suffering from heart failure, it can be decided whether a subject will be susceptible for a therapy as referred to above. Specifically, it is envisaged that a subject having an amount of GDF-15 larger than the reference amount will be suitable to be treated by the aforementioned therapy while a subject with less GDF-15 will not benefit from the therapy.

Encompassed by the present invention is, further, a device adapted to carry out the methods of the present invention, comprising means for determining the amount of GDF-15 in a sample of the subject and means for comparing said amount to a reference amount, whereby a type 1 diabetes patient having a predisposition for the complications as specified beforehand is identified.

The term “device” as used herein relates to a system of means comprising at least the aforementioned means operatively linked to each other as to allow the prediction. Preferred means for determining the amount of GDF-15, and means for carrying out the comparison are disclosed above in connection with the method of the invention. How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. Alternatively, where means such as test stripes are used for determining the amount of the peptides or polypeptides, the means for comparison may comprise control stripes or tables allocating the determined amount to a reference amount. The test stripes are, preferably, coupled to a ligand which specifically binds to the peptides or polypeptides referred to herein. The strip or device, preferably, comprises means for detection of the binding of said peptides or polypeptides to the ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test stripes or electronic devices which merely require loading with a sample. The results may be given as output of raw data which need interpretation by the clinician. Preferably, the output of the device is, however, processed, i.e. evaluated, raw data the interpretation of which does not require a clinician. Further preferred devices comprise the analyzing units/devices (e.g., biosensors, arrays, solid supports coupled to ligands specifically recognizing the peptide, Plasmon surface resonance devices, NMR spectrometers, mass-spectrometers etc.) or evaluation units/devices referred to above in accordance with the method of the invention.

Accordingly, the present invention also relates to a device for predicting if a diabetes type 1 patient will suffer from one or more complications selected from cardiovascular complications, terminal renal failure, and death, comprising means for determining the amount of GDF-15 in a sample of the subject and means for comparing said amount to a reference amount.

Further envisaged is a device for assessing the risk of a diabetes type 1 patient to suffer from one or more complications selected from cardiovascular complications, terminal renal failure, and death, comprising means for determining the amount of GDF-15 in a sample of the subject and means for comparing said amount to a reference amount.

The present invention also relates to a device for deciding on the administration of medicaments in a diabetes type 1 patient being susceptible to suffer from a cardiovascular complication, terminal renal failure, and/or death, comprising means for determining the amount of GDF-15 in a sample of the subject and means for comparing said amount to a reference amount.

Furthermore, the present invention encompasses a kit adapted to carry out the methods of the present invention, comprising means for determining the amount of GDF-15 in a sample of the subject and means for comparing said amount to a reference amount, whereby a type 1 diabetes patient having a predisposition for the complications as specified beforehand is identified.

The term “kit” as used herein refers to a collection of the aforementioned means, preferably, provided in separately or within a single container. The container, also preferably, comprises instructions for carrying out the method of the present invention.

The present invention pertains to a kit for predicting if a diabetes type 1 patient will suffer from one or more complications selected from cardiovascular complications, terminal renal failure, and death, comprising means for determining the amount of GDF-15 in a sample of the subject and means for comparing said amount to a reference amount.

Also, the present invention relates to a kit for assessing the risk of a diabetes type 1 patient to suffer from one or more complications selected from cardiovascular complications, terminal renal failure, and death, comprising means for determining the amount of GDF-15 in a sample of the subject and means for comparing said amount to a reference amount.

Finally, the present invention relates to a kit for deciding on the administration of medicaments in a diabetes type 1 patient being susceptible to suffer from a cardiovascular complication, terminal renal failure, and/or death, comprising means for determining the amount of GDF-15 in a sample of the subject and means for comparing said amount to a reference amount.

All references cited in this specification are herewith incorporated by reference with respect to their entire disclosure content and the disclosure content specifically mentioned in this specification.

The following Example shall merely illustrate the invention. It shall not be construed, whatsoever, to limit the scope of the invention.

### Example 1

GDF-15 is a predictor for an increased risk of death and developing renal failure in patients suffering diabetes mellitus type 1.

A total of 891 patients suffering from diabetes type I were investigated for blood levels of GDF-15. Blood levels GDF-15 levels were determined by a third-generation assay on an ELECSYS 2010 analyzer (Roche Diagnostics).

Endpoints “all cause mortality” and “renal failure” were determined after 12 years in the present outcome study.

The results of the study are summarized in the following table.

TABLE 1

| | | all cause | end stage | fatal and non- | fatal | non fatal |
|---|---|---|---|---|---|---|
| GDF-15 | | mortality | renal disease | fatal cv-event | cv-event | cv-event |
| [pg/ml] | percentile | N = 178 | N = 89 | N = 211 | N = 78 | N = 133 |
| 698 | 25^{th} | 4 | 1 | 26 | 1 | 25 |
| 974 | 50^{th} | 21 | 1 | 31 | 6 | 25 |
| 1467 | 75^{th} | 39 | 5 | 51 | 15 | 36 |
| 3567 | 95^{th} | 114 | 82 | 103 | 56 | 47 |

N = 891 patients
N = 178 patients with all cause mortality
N = 89 patients with end stage renal disease ERSD
N = 211 patients with fatal and non-fatal cardiovascular events
N = 78 patients with fatal cardiovascular (cv) events
N = 133 patients with non fatal cardiovascular (cv) event

What is claimed is:

1. A method for predicting susceptibility of terminal renal failure in a type 1 diabetes patient, the method comprising:
determining an amount of growth-differentiation factor 15 (GDF-15) in a sample from the patient, and
comparing the amount of GDF-15 determined to a reference amount of GDF-15 wherein a prediction of susceptibility of terminal renal failure in the subject is indicated when the determined amount of GDF-15 is greater than the reference amount.

2. The method claim 1, wherein the reference amount for GDF-15 is 1500 pg/ml.

3. The method of claim 1, wherein the reference amount is 2000 pg/ml.

4. The method of claim 1, wherein the reference amount for GDF-15 is 2500 pg/ml.

5. A method for assessing a risk of a diabetes type 1 patient suffering from terminal renal failure, the method comprising:
determining the amount of GDF-15 in a sample from a diabetes type 1 patient and
comparing the amount of GDF-1.5 determined to a reference amount of GDF-15 wherein a risk of terminal renal failure in the patient is indicated when the determined amount of GDF-15 is greater than the reference amount.

6. A method for deciding on administration of medicament in a diabetes type 1 patient who has a susceptibility of terminal renal failure, the method comprising
determining an amount of growth-differentiation factor 15 (GDF-15) in a sample from the patient, and
comparing the amount of GDF-15 determined to a reference amount of GDF-15 wherein a decision for administration of medicament is indicated when the determined amount of GDF-15 is greater than the reference amount.

7. A device for predicting susceptibility of terminal renal failure in a type 1 diabetes patient according to the method of claim 1, the device comprising
means for determining an amount of growth-differentiation factor 15 (GDF-15) in a sample from the patient and
means for comparing the amount of GDF-15 determined to a reference amount of GDF-15.

8. A kit for predicting susceptibility of terminal renal failure in a type 1 diabetes patient according to the method of claim 1, the kit comprising
means for determining an amount of growth-differentiation factor 15 (GDF-15) in a sample from the patient and
means for comparing the amount of GDF-15 determined to a reference amount of GDF-15.
